(19) United States
(12) Patent Application Publication
(10) Pub. No.: US 2002/0049188 A1
(43) Pub. Date: Apr. 25, 2002

(54) Nitroglycerin ointment for treatment of pain associated with anal disease

(76) Inventors: Daniel L. Azarnoff, Hillsborough, CA (US); Charles E. Lee, Union City, CA (US); Jung-Chung Lee, San Jose, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/738,120

(22) Filed: Dec. 15, 2000

Related U.S. Application Data
(60) Provisional application No. 60/172,292, filed on Dec. 15, 1999.
(60) Provisional application No. 60/176,219, filed on Jan. 14, 2000.
(60) Provisional application No. 60/214,043, filed on Jun. 23, 2000.

(51) Int. Cl. A61K 31/5685; A61K 31/21

(52) U.S. Cl. 514/171; 514/509

(57) ABSTRACT

The present invention provides methods for reducing pain associated with chronic anal fissures with reduced side effects, comprising: contacting an anal area with a composition comprising an effective amount of nitroglycerin, thereby relieving pain associated with anal fissures. It also provides a extremely stable pharmaceutical composition comprising nitroglycerin, propylene glycol, and a non-ionic surfactant, as well as kits.

### CROSS-REFERENCE TO RELATED APPLICATIONS

This application claims priority to U.S. Patent Application No. 60/172,292, filed Dec. 15, 1999; U.S. Patent Application No. 60/176,219, filed Jan. 14, 2000; and U.S. Patent Application No. 60/214,043, filed Jun. 23, 2000; the teachings of which are each incorporated by reference in their entirety for all purposes.

### FIELD OF THE INVENTION

The present invention relates to the treatment of anal diseases. More particularly, the present invention provides methods of reducing pain associated with anal diseases using a nitric oxide donor composition.

### BACKGROUND OF THE INVENTION

In general, anal fissure (fissure-in-ano), anal ulcer, acute hemorrhoidal disease, and levator spasm (proctalgia fugax) are common, benign conditions of the anal canal which affect humans of all ages, races and sexes. However, these conditions can be problematical to treat and inconvenient if not painful to endure.

An anal fissure or ulcer is a tear or ulcer of the mucosa or lining tissue of the distal anal canal. Anal fissure/ulcer can be associated with other systemic or local diseases, but is more frequently present as an isolated finding. The typical, idiopathic fissure or ulcer is confined to the anal mucosa, and usually lies in the posterior midline, distal to the dentate line. A person with an anal fissure or ulcer frequently experiences anal pain and bleeding, the pain being more pronounced during and after bowel movements.

Hemorrhoids are specialized vascular areas lying subjacent the anal mucosa. Symptomatic hemorrhoidal disease is manifest by bleeding, thrombosis and/or prolapse of the hemorrhoidal tissues. Commonly, internal hemorrhoidal tissue bulges into the anal canal during defecation causing bleeding and pain. As the tissue enlarges, further bleeding and pain, prolapse and thrombosis can ensue. The thrombosis of hemorrhoids is another cause of bleeding and pain.

Levator spasm is a condition affecting women more frequently than men. This syndrome is characterized by spasticity of the levator ani muscle, a portion of the anal sphincter complex. A patient suffering from levator spasm may experience severe, episodic rectal pain. Physical exam may reveal spasm of the puborectalis muscle and pain may be reproduced by direct pressure on this muscle. Bleeding is normally not associated with this condition.

The underlying causes of these anal disorders are poorly understood, but all of these conditions are associated with a relative or absolute degree of anal sphincter hypertonicity. In the case of anal fissure/ulcer, the abnormality appears to be an as-yet-unidentified problem of the internal anal sphincter muscle. The internal sphincter is a specialized, involuntary muscle arising from the inner circular muscular layer of the rectum. Intra-anal pressure measurements obtained from people suffering from typical anal fissure/ulcer disease show an exaggerated pressure response to a variety of stimuli.

Various therapies have been devised to treat these anal disorders. Typical, non-surgical therapy includes bulk laxatives and sitz baths. Sitz baths are helpful because they induce relaxation of the anal sphincter mechanism (see, e.g., Shafik, “Role of warm-water bath in anorectal conditions: The ‘thermosphincteric reflex,’” J. Clin. Gastroenterol. 16:304-308 (1993)).

Topical anal therapy is also used in an effort to promote healing, relieve pain, and reduce swelling and inflammation. Many preparations have been tried including those containing local anesthetics, corticosteroids, astringents, and other agents. However, none of these preparations has been shown conclusively to reduce the healing time or to reliably ameliorate associated pain. More particularly, corticosteroids such as hydrocortisone have shown some benefit thereby, but not in a reproducible nor significant fashion. Furthermore, any relief from anal pain due to topical anesthetics such as dibucaine, lidocaine, pramoxine has been relatively short-lived.

In certain instances, surgery may be employed to treat anal disorders. Cases of anal fissure/ulcer or hemorrhoids recalcitrant to medical therapy are often referred for surgical treatment. In keeping with the proposed etiology of anal fissure/ulcer, the current standard surgical procedure therefore is lateral internal anal sphincterotomy. In this procedure, the internal anal sphincter muscle is partially cut, thereby reducing the intra-anal pressure. The surgery is well tolerated, but requires anesthesia and in a small number of patients results in incontinence. Surgical hemorrhoidectomy removes the redundant hemorrhoidal tissue, and many surgeons will perform concomitant limited internal anal sphincterotomy to lower anal canal pressure. A significant drawback to surgical hemorrhoidectomy is that the area upon which the surgery was performed remains quite painful for an extended period of time following the surgery. There is no successful surgical treatment for levator spasm.

Nitric oxide (NO), an inhibitory neurotransmitter to muscle, interacts with the enteric nervous system to regulate the motor and secretory function of the gut continuously from the esophagus to anus. In particular, NO has been shown to mediate the anorectal inhibitory reflex in animals and humans.

Recently, Lund and Scholefield (see, Lund et al., Randomised, double-blind, placebo-controlled trial of glyceryl trinitrate ointment in the treatment of anal fissure. Lancet 349: p. 11-14 (1997)) published a prospective, double blind, randomized trial of NTG ointment versus placebo, providing further evidence that NTG promotes the healing of chronic anal fissures. Patients were randomized to groups using 500 mg of 0.2% NTG ointment or placebo twice daily for eight weeks. In this study, anal fissures healed in 26/38 (68%) patients treated with NTG ointment compared to 3/39 (8%) treated with placebo, p<0.0001. Pain was significantly reduced after two weeks of treatment with NTG whereas there was no reduction in pain experienced by those subjects treated with placebo.

Bacher et aL (see, Bacher et al., Local nitroglycerin for treatment of anal fissures: An alternative to lateral sphincterotomy? Dis. Colon Rectum 40:840-845 (1997)) studied 35 patients with acute and chronic anal fissures in a randomized, double blind study comparing 0.2% NTG ointment to 2% lidocaine gel. Eighty percent of the NTG group healed after four weeks of therapy with NTG ointment compared to 60% of those treated with lidocaine gel (p=<0.01). Five of the eight patients (62.5%) treated with NTG ointment healed in four weeks compared to only one of five (20%) treated with lidocaine gel. Pain was significantly reduced after two weeks and/of treatment with NTG ointment. Significantly, headache occurred in 20% of patients treated with NTG ointment.

U.S. Pat. No. 5,696,676 (“Gorfine et al.”) describes application of 500 to 1000 mg of 0.5% nitroglycerin compositions that markedly reduce pain and promote healing of anal fissures. Significantly, over 33% of subjects experienced headaches.

As indicated by the studies described above, headaches are a fairly common side effect of administration of nitroglycerin compositions for treatment of anal fissures. Remarkably, in certain studies the incidence of headaches is as high as 60-80% (see, Hasegawa et al., Ann. R. Coll. Surg. Engl. 82:27-30 (2000); Hyman et al. Dis. Colon Rectum 42:383-5 (2000)). The need for finding a balance between efficacy for treatment of anal fissures and headache incidence is widely recognized as a significant clinical issue (see, Palazzo et al, J.R. Coll. Surg. Edinb. 45:168-70 (2000)).

Moreover, in previous studies the exact amount of ointment and thus nitroglycerin applied to the anal area was not carefully controlled. As a consequence, it is not known what exact dose of nitroglycerin provides significant pain relief.

Therefore, despite the advances of Gorfine et al. and others, there remains a need in the art for effective relief of the pain of anal disorders, especially anal fissures, with reduced side effects. Dosages and concentrations of nitroglycerin compositions are needed to relieve pain arising from anal conditions with reduced side effects. The present invention fulfills these and other needs.

### SUMMARY OF THE INVENTION

Headaches are a common side effect of administration of nitroglycerin compositions. In addition, formulating nitroglycerin into a suitable topical form is a difficult endeavor. The present invention relates to methods for using nitroglycerin compositions to relieve pain associated with an anal disease with minimal side effects. Other therapies with nitroglycerin compositions have been demonstrated to relieve pain, but do so with significant side effects.

As such, in one aspect, the present invention provides a method for relieving pain in a mammal associated with an anal condition with a dramatic decrease in side effects, the method comprising contacting an anal area with a composition comprising nitroglycerin in an amount of 0.4% by weight. Advantageously, it has been discovered that administration of a 0.4% by weight nitroglycerin composition is extremely well tolerated (e.g., a dramatic decrease in side effects such as headaches). The number of patients experiencing reduced side effects or no headaches is greater than about 60%-80%, more preferably, about 80-90%, and most preferably, about 96%. The mammal is preferably a human being.

Remarkably, administration of a pharmaceutical composition comprising 0.4% by weight of nitroglycerin is extremely efficacious in relieving pain from anal conditions and diseases with a concomitant decrease in side effects. Moreover, the composition of the present invention has been found to be extremely stable remaining as a single phase after 3-months of storage.

In yet another embodiment, the composition further comprises an effective amount of corticosteroid. In certain preferred embodiments, the corticosteroid is present in an amount of from about 0.001% to about 10% by weight, based upon the total weight of the composition. Suitable corticosteroids include, but are not limited to, hydrocortisone, cortisol, and dexamethasone phosphate.

In still another embodiment, the composition further comprises an effective amount of an anesthetic. In certain preferred embodiments, the anesthetic is present in an amount of from about 0.1% to about 10% by weight, based upon the total weight of the composition. Suitable anesthetics, include, but are not limited to, dibucaine, lidocaine, pramoxine, benzocaine, and tetracaine. Preferably, the composition is adapted for topical administration to the external anus and anal canal. In still yet another embodiment, the composition comprises one or more pharmaceutically acceptable carriers or excipients in admixture with the nitroglycerin. In certain preferred embodiments, the excipients are white petrolatum, mineral oil, lanolin, distilled water, acetone sodium bisulfite, zinc oxide, or cocoa butter. In a particularly preferred embodiment, the composition comprises nitroglycerin, propylene glycol, and a non-ionic surfactant. Preferably, the composition is in a suitable topical form, such as a powder, an aerosol, a liquid, a thickened liquid, a foam, a tablet, a capsule, a semi-solid, an emulsion, an ointment, a gel, a cream, or a suppository.

In another aspect, the present invention provides a method for relieving pain in a mammal associated with an anal disease with a concomitant decrease in side effects, the method comprising contacting an anal area with a composition comprising nitroglycerin in an amount of 150 milligrams. The composition was extremely well tolerated. The number of patients experiencing reduced side effects or no headaches is greater than about 60%-80%, more preferably, about 80-90%, and most preferably, about 96%. The mammal is preferably a human being.

The compositions and methods of present invention are also useful in treating conditions resulting from spasms and/or hypertonicity of sphincters of the anorectal region including anal fissure, post-operative rectal pain, hypertrophic pyloric stenosis, and pancreatitis, as well as conditions resulting from general spasm of the muscles of the GI tract including Zenkers diverticulum, achalasia, esophageal spasm (nutcracker esophagus), irritable bowel disease, and Hirshprungs disease (bowel obstruction).

In yet another aspect, the present invention is also useful in reducing or relieving pain following surgical procedures, such as post-hemorrhoidectomy pain. In addition, the present invention is useful for relaxing the anal sphincter and reducing pain before, during and after examinations of the anus, rectum and lower gastrointestinal system, insertion of instruments, and procedures, such as surgery.

In still other aspects, the present invention provides unit dose containers of the composition, e.g. aluminum pouches, etc. The present invention also provides kits with the composition in a suitable container, directions for use, a measuring device, and optionally an applicator.

Further combinations, compositions, and aspects of the present invention will be apparent when read with the following detailed description.

### BRIEF DESCRIPTION OF THE DRAWINGS

FIG. 1 illustrates the healing probability for chronic anal fissures treated with different compositions of nitroglycerin applied twice a day.

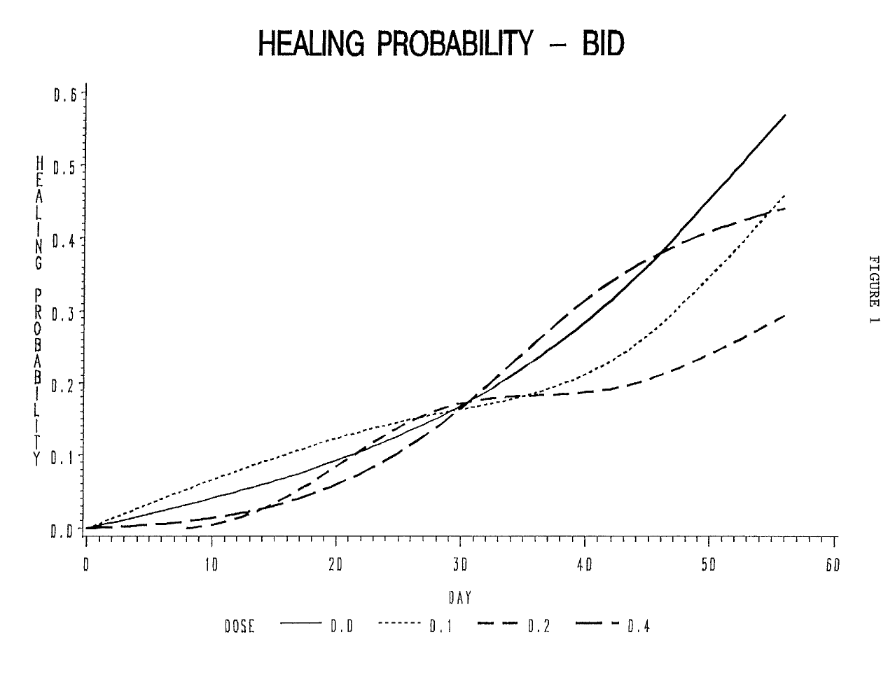

FIG. 2 illustrates the healing probability for chronic anal fissures treated with different compositions of nitroglycerin applied three times a day.

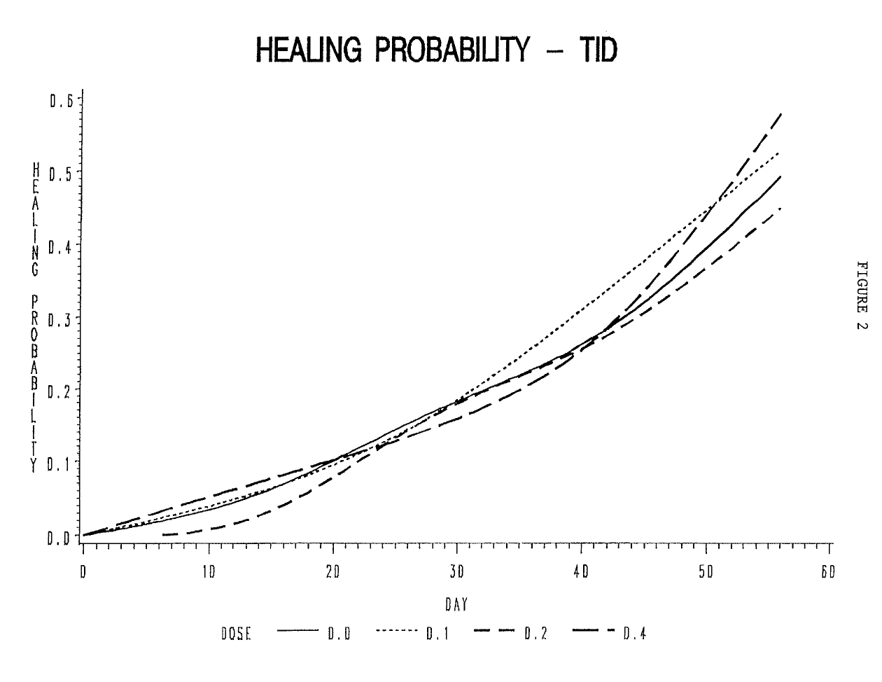

FIG. 3 illustrates the mean percent average pain reduction for subjects with chronic anal fissures treated with different compositions of nitroglycerin.

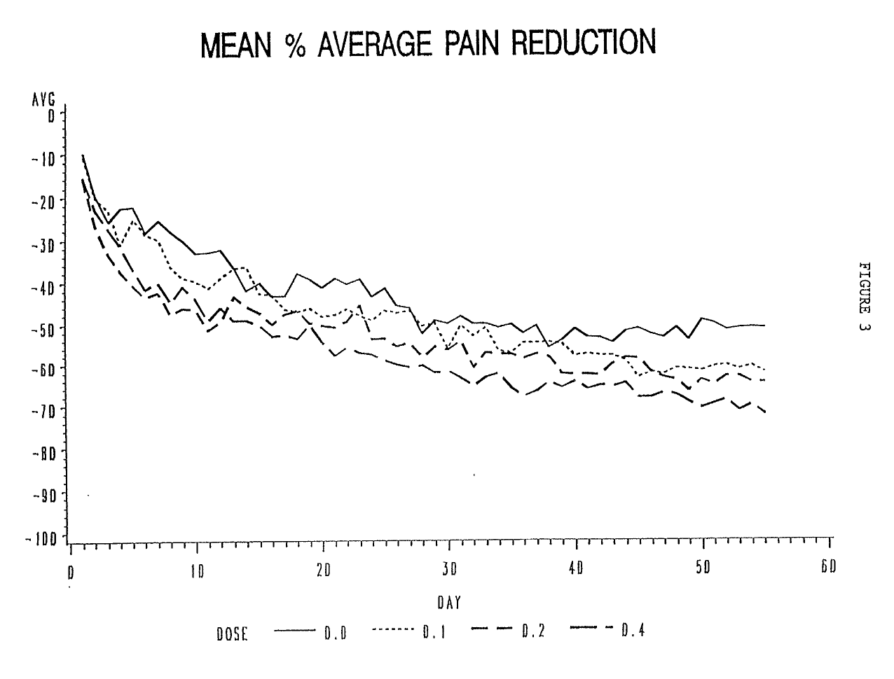

FIG. 4 illustrates the mean percent defecation pain reduction for subjects with chronic anal fissures treated with different compositions of nitroglycerin.

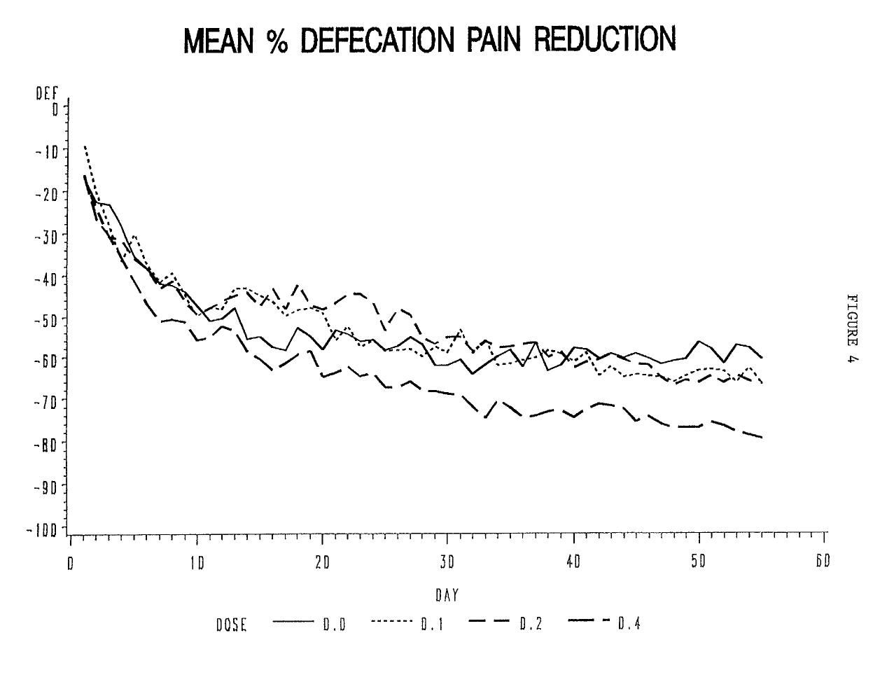

FIG. 5 illustrates the mean percent worst pain reduction for subjects with chronic anal fissures treated with different compositions of nitroglycerin.

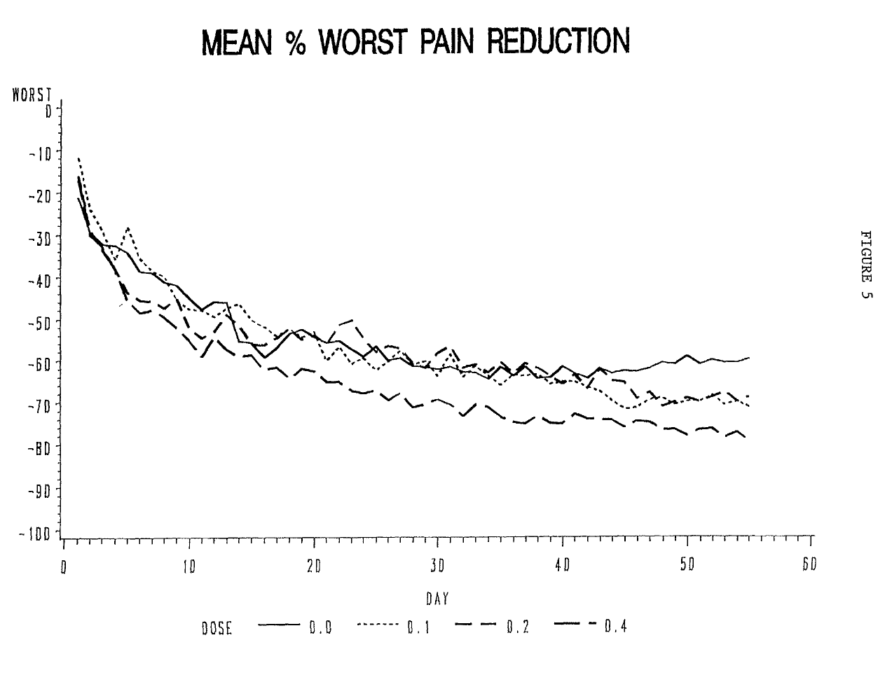

### DETAILED DESCRIPTION OF THE INVENTION AND PREFERRED EMBODIMENTS

I. Definitions

The term “anal” includes musculature and vasculature tissue of or proximate the anus and/or lower gut.

The term “anal disease” means a disorder of the tissue which can include musculature and/or vasculature of or proximate the anus and/or lower gut.

The term “organic nitric oxide donor” means an organic compound or mixture of compounds with at least one of such compound(s), which can release nitric oxide under physiological or anal disease treatment conditions.

The term “pharmaceutically-acceptable” as used herein refers to pharmaceutical actives (or permeants), as well as the other compatible drugs, medications or inert ingredients which are suitable for use in contact with the tissues of humans and animals without undue toxicity, irritation, allergic response, and the like commensurate with a reasonable benefit/risk ratio.

The term “topical administration” as used herein refers to directly or optionally indirectly, laying or spreading upon tissue, such as epithelial tissue, especially outer skin or other biological membranes, including the skin or membrane of the anal or other epithelial and mucosal cavities.

The term “anal area” refers to any area or tissue in and around the anus or internal anal sphincter or external anal sphincter which is affected by or subject to anal disorder or disease, or where application of compositions and methods of the invention will exert the largest therapeutic effect. This area varies according to the particular condition to be treated. For example, for an anal ulcer, the composition is preferably applied to the distal anal canal. For hemorrhoids, the composition is preferably applied to the hemorrhoidal area itself, the vascular areas subjacent to the anal mucosa. For levator spasm, the composition is preferably applied to the levator ani muscle, a portion of the anal sphincter complex. For other diseases and conditions, the appropriate anal area is the anal verge, the external anus, the internal anal canal, the proximate anal canal, the external or internal anal sphincter or anal sphincter muscle and combinations thereof.

II. Pharmaceutical Compositions Used in the Methods

The present invention concerns a treatment directed at anal disease arising from an abnormality of the anal sphincter muscles. Preferably, the invention is directed to anal ulcers, hemorrhoids, and levator spasm, and most preferably, towards anal chronic fissure. The treatment according to the invention comprises application of an effective amount of a nitric oxide donor, preferably nitroglycerin, to afflicted tissue.

As its efficacy has been demonstrated in clinical trials, in preferred embodiments the nitric oxide donor is nitroglycerin. In certain aspects, the present invention provides a dose of nitroglycerin (NTG) ointment that relieves the pain of chronic anal fissures. In a study of the present invention, a randomized double-blind study of locally applied NTG ointment (Anogesic®, Cellegy Pharmaceuticals) was conducted with patients with chronic anal fissures. The patients were randomized to one of the eight treatment regimens (0.0, 0.1, 0.2, 0.4% NTG applied twice a day (b.i.d) or three times a day (t.i.d)), for up to eight weeks. A dose-measuring device standardized the delivery of 375 mg ointment (see, Example).

Healing of fissures (complete re-epithelialization) was assessed by physical examination utilizing an observer unaware of treatment allocation. The results indicated that treatment with 0.4% NTG ointment was associated with a highly significant (p<0.0002) decrease in pain intensity assessed by patients with a visual analog scale. The decreases were observed within the first week of treatment. Treatment was well tolerated. Surprisingly, only 3.6% of patients discontinued treatment due to headache (96% of the patients experienced no side effects or experiencing well toleration). This remarkably low incidence of side effects is in contrast to previously studied nitroglycerin compositions, which provide significant pain reduction and healing, but also result in headaches in a substantial proportion of subjects. In particular, it appears that a 20% reduction in concentration from 0.5%, the nitroglycerin composition used in Gorfine et al., to 0.4%, results in an unexpectedly dramatic reduction in side effects. Thus, the present invention provides a nitroglycerin (NTG) composition to reduce the pain of chronic anal fissures with minimal side effects.

Without being bound by any particular theory, the unexpectedly dramatic reduction in side effects upon using the compositions and methods of the present invention is believed to relate to a cascade mechanism involving activation of an optimum amount of solubilized guanylate cyclase and concomitant effectuation of vascular smooth muscle. Alternatively, it may be due to an identification of the optimal balance in maximizing the local effect and minimizing the systemic side effects exerted by the current composition.

Therefore, in preferred embodiments the nitroglycerin is present at about 0.4% by weight. In an alternative embodiment, 150 mg nitroglycerin or 375 mg of 0.4% nitroglycerin ointment is administered.

The invention also provides the composition in a suitable topical or suppository physiologically acceptable carrier, optionally complexed with other agent(s) such as a corticosteroid and/or a topical anesthetic.

Employment of the optional corticosteroid and/or topical anesthetic in the practice of the invention can provide decidedly advantageous results. In cases where treatment with a nitric oxide donor alone as active treating agent fails to provide sufficient relief from pain and/or healing, most notably, the employment of the corticosteroid and/or topical anesthetic in combination with the nitric oxide donor often can provide significantly enhanced if not complete relief from pain and provide for significant if not total healing as well.

Optionally, a corticosteroid can be present in the compositions of the present invention. For instance, the corticosteroid can include hydrocortisone, i.e., 11-17-21-trihydroxypregn-4-ene-3,20-dione or cortisol, cortisol acetate, hydrocortisone phosphate, hydrocortisone 21-sodium succinate, hydrocortisone tebutate, corticosterone, corticosterone acetate, cortisone, cortisone acetate, cortisone 21B-cyclopentanepropionate, cortisone phosphate, triamcinolone hexacetonide, dexamethasone phosphate, desonide, betamethasone dipropionate, mometasone furate, and so forth and the like.

In general, the corticosteroid can be present in any amount, which is effective in the practice of the treatment of anal disease. In typical practice of the invention, the corticosteroid can be present in a concentration from about 0.001 to about 10 percent by weight and preferably from about 0.1 to about 5 percent by weight. If cortisol is the corticosteroid, preferred concentrations reside in the range of from about 0.1 to about 2.5 percent by weight. If hydrocortisone is the corticosteroid, preferred concentrations reside in the range of from about 0.5 to about 5 percent by weight. If dexamethasone phosphate is the corticosteroid, preferred concentrations reside in the range of from about 0.005 to about 0.03 percent by weight.

Optionally, a topical anesthetic can be present in the composition of the invention. For instance, the topical anesthetic can include dibucaine, lidocaine, pramoxine, benzocaine, tetracaine, and so forth and the like. In general, the topical anesthetic can be present in any amount, which is effective in the practice of the treatment of anal disease. In typical practice of the invention, the topical anesthetic can be present in a concentration from about 0.1 to about 5 percent by weight and preferably from about 0.5 to about 4 percent by weight based on the total weight of the composition. If dibucaine is the topical anesthetic, preferred concentrations reside in the range of from about 0.25 to about 2 percent by weight. If benzocaine is the topical anesthetic, preferred concentrations reside in the range of from about 10 to about 20 percent by weight. If tetracaine is the topical anesthetic, preferred concentrations reside in the range of from about 1 to about 2 percent by weight. The corticosteroid and topical anesthetic can be employed together in the practice of the invention.

Optionally, additional pharmacologic agents can be present in the composition. Representative compositions include topical compositions comprising nitroglycerin in an acceptable carrier and at least one of the following additional pharmacologic agents: a local anesthetic (e.g., lidocaine, prilocaine, etc.), local anti-inflammatory agent (e.g., naproxen, pramoxicam, etc.), corticosteroid (e.g., cortisone, hydrocortisone, etc.), anti-itch agent (e.g., loperamide, diphylenoxalate, etc.), an agent that interferes with the activation of peripheral sensory neurons, including divalent and trivalent metal ions (e.g., manganese, calcium, strontium, nickel, lanthanum, cerium, zinc, etc.), yeast-based product (e.g., lyophilized yeast, yeast extract, etc.), growth-promoting and/or wound healing-promoting agent known to promote re-epithelialization (e.g., platelet-derived growth factor or PDGF, etc.), anti-microbial agent (e.g., neosporin, polymyxin B sulfate, bacitracin zinc, etc.), mucoadhesive agent (e.g., cellulose derivatives, etc.), cytoprotectant agent (e.g., colloidal bismuth, misoprostol, sucralfate, etc.) as defined in Goodman & Gilman's The Pharmacological Basis of Therapeutics, supra, an agent that promotes local tissue sclerosis (e.g., alum, etc.), menthol, or eucalyptol.

II. Formulation

As those skilled in the art can appreciate, the composition of the invention can be formulated in any pharmaceutical state suitable for topical application, examples of which include liquid, aerosol, thickened liquid, emulsion, semisolid, foam, powder, and a tablet or capsule, which can be lubricated for insertion into the anus. The method of the invention can employ any of such formulations as may be appropriate for treatment in particular cases. Advantageously, the composition can be formulated into highly convenient dosage forms with thickening agents to include thickened solutions or lotions, ointments to include creams and gels, and so forth.

Thickened solutions or lotions and ointments can be formed by incorporating with the active ingredients various gelling agents or other thickeners (viscosity increasers) which permit release of the active ingredients to the skin or tissue upon or following application. These forms are advantageously employed to lessen the runoff from the skin or tissue, which can occur with more fluid (less viscous) formulations. Importantly, they also permit more sustained contact of the active ingredient(s) and any penetration enhancer with the treated surfaces, thus permitting an enhancement of the speed of delivery of the active ingredient(s) subcutaneously, and providing more accurate and controllable dosing. Accidental spilling and undesired contact with the composition can also be minimized with such types of formulations.

It can be advantageous to employ water-dispersible thickening agents, i.e., agents dispersible in water to form a homogeneous distribution or even solution, such as the polyethylene glycols and similar agents, as they are readily compatible with water or other diluents which can be formulated in the composition. Alternatively, an emulsion base can be employed to impart the desired thickening effect, together with the emollient effect of the lipoid phase of the emulsion base.

Water-soluble or water-dispersible thickening bases or substances can employ polyethylene glycols and the like of different viscosities depending upon the desired consistency and concentration of active ingredient(s) which can be incorporated into the composition. Other thickening agents which can be suitable for employment herein include but are not limited to water-dispersible gums, carboxyvinyl polymers, methyl cellulose, sodium carboxymethyl cellulose, and alginates.

Lotions and ointments incorporating emulsion bases can contain the usual ingredients to provide the base, including fatty alcohols such as acetyl alcohol, an emulsifier such as, for example, lauryl sulfate, and water. Also, the remainder of a topical preparation can contain one or more conventional ointment components such as, for example, white petrolatum, lanolin, distilled water, and mineral oil in conventional amounts. The remainder of a suppository can contain conventional amounts of known suppository components such as, for example, zinc oxide and/or cocoa butter.

Sustained or Controlled Delivery Formulations

In yet other embodiments, the invention provides topical sustained and prolonged release pharmaceutical compositions comprising nitroglycerin to treat anorectal disorders and the pain associated therewith. Topical sustained and prolonged release compositions are typically variants which include 1) an absorbent in a hydrophilic base; 2) an absorbent in a hydrophobic base; and 3) coated beads containing an absorbent matrix dispersed in a suitable vehicle. Also provided are methods of treating anal or GI tract disorders comprising topically administering an effective amount of such compositions to the appropriate anal area of the subject in need of such treatment.

Such hydrophilic compositions and preparations of the invention comprise nitroglycerin and a polymer, such as cellulose (methyl cellulose, ethyl cellulose, hydroxy propyl cellulose, etc.), higher molecular weight polyethylene glycol, methacrylic-acrylic acid emulsion, hydrogel, carbopol, ethyl vinyl acetate copolymer, or polyester, etc., to bind the nitroglycerin to the polymer. The nitroglycerin-polymer is then dispersed in a hydrophilic vehicle to form a semi-solid. After administration of such hydrophilic composition into the appropriate anal area, such as the anal canal or anal sphincter, the water in the semi-solid preparation is adsorbed and the polymer matrix with the nitroglycerin remains as a coating in the anal region or area to which it has been applied. The nitroglycerin is then slowly released from this coating.

Hydrophobic compositions and preparations of the inventions employ similar polymers as used in the hydrophilic preparations, but the polymer/nitroglycerin matrix is dispersed into a vehicle, such a plastibase, in the hydrophobic compositions and preparations. Plastibase is a mineral oil base that only partially dissolves the nitroglycerin. The semi-solid composition forms a thin coating on the anal region to which the composition has been applied (such as the anal canal or anal sphincter area) and slowly releases the active. The prolonged action is controlled principally by the solubility of the active ingredient (nitroglycerin) in the vehicle.

The present invention also provides coated beads which are produced by first absorbing the nitroglycerin on a cellulosic material blended with polyethylene glycol, filler, binder and other excipients. The resulting matrix is then extruded and spheronized (e.g., the process of making into spheres) to create small beads. The beads are then coated to an appropriate thickness with one or more a suitable material, such as a methacrylic-acrylic polymer, polyurethane, ethyl vinyl acetate copolymer, polyester, silastic, etc. The coating on the beads acts as a rate controlling membrane, which regulates the release of the nitroglycerin from the core beads.

Current nitroglycerin ointment products on the market consist of nitroglycerin adsorbed onto lactose dispersed in a petrolatum/lanolin base. These products are difficult to manufacture and NTG on lactose can be an explosive hazard. In addition, these products are physically unstable at temperatures near 40° C. This invention provides a pharmaceutical composition of nitroglycerin with an alternative excipient, propylene glycol. Nitroglycerin dissolved in propylene glycol has not been traditionally used to manufacture ointments since the nitroglycerin-propylene glycol solution is not miscible with the standard ointment base. Surprisingly, the use of a non-ionic surfactant, such as sorbitan sequioleate, forms an extremely stable emulsion with NTG in propylene glycol and petrolatum and petrolatum-lanolin mixtures. Such ointment formulations are significantly more stable than previous ointment formulations of nitroglycerin, particularly at elevated temperatures.

The preferred embodiment of this invention, a 0.4% nitroglycerin by weight composition, is physically stable for up to three months. The use of propylene glycol, rather than lactose, also makes this formulation easier to process and less of an explosive hazard. Therefore, in preferred embodiments, the composition of the present invention comprises propylene glycol and a non-ionic surfactant. These surfactants include, but are not limited to, sorbitan sesquioleate, sorbitan monostearate, propylene glycol monolaurate, sorbitan mono-oleate, glycerol monostearate, propylene glycol monostearate, sorbitan tristearate, and sorbitan trioleate. Such formulations are monophasic for long periods of time and do not separate at all during 3-months of storage at elevated temperatures, such as those used under accelerated stability storage conditions. The formulations remain homogeneous (monophasic) during storage.

III. Dosages and Methods of Administration

Pourable pharmaceutical dosages can be provided and dispensed in graduated containers, or in containers which contain a given volume, say, for example, 5 or 10 cc, and so forth. Containers with greater volumes, say, for example, of 20 cc and greater, can provide convenient multiple dosage forms. Containers containing a typical single dose, such as aluminum pouches, can provide convenient dosage forms. Squeeze tubes for lotions and ointments and cotton stick applicators can be employed for topical application of the composition for liquids ranging from those of water-like viscosity to the more viscous formulations of thickened compositions and for powders and the like.

In one embodiment, the present invention also relates to providing the nitroglycerin composition in kit form. In preferred embodiments, the kit comprises a nitroglycerin composition, a container for containing the composition, directions for the administration of the composition, a dose-measuring device, and optionally an applicator (e.g., a finger cot for application to the distal anus). The kit form is particularly advantageous for ease of use and proper administration outside of a controlled clinical environment. The composition is preferably 0.4% nitroglycerin. Optionally, each item of the kit is self-contained within the container.

The composition of the invention, and the ingredient(s) in a method, can also be administered by dusting, spraying or misting such as from shakers, dusting devices, misting devices and aerosol bottles. Containers of the composition can be charged with a suitable amount and concentration of ingredient(s). As an illustration, a container can be charged with a fluid formulation, along with an aqueous diluent, optionally with thickening agent(s), physiological salt(s), and so forth. Liquid compositions, for example, can be administered as low viscosity substances to semisolid gels or mousses, depending on any amount of gelling agent(s) and/or surfactant(s) included therein. Such compositions can be sufficiently fluid to permit their dispensing by spray or mist from the container and also can meet criteria for penetrability. Dusts can be employed. An inert ingredient such as, for example, starch and/or talc can be employed to dilute the active ingredient(s) in powder form.

In treatment according to the invention, an amount of active ingredient(s) or composition of the invention is contacted with or applied to the affected anal area or proximate thereto such that an effective amount of nitric oxide, preferably delivered by release from an organic nitric oxide donor, is administered. The amount of active ingredient(s) or composition, which is employed, should be effective for the amelioration, control and/or healing of the anal disease and the prompt and dramatic control or relief of pain resulting from or associated with the disease.

The present invention further provides compositions in a pharmaceutically acceptable dosage form useful in treating medical conditions such as hemorrhoidal pain and for treating spasms and/or hypertonicity of the sphincters including the internal anal sphincter, lower esophageal sphincter, pyloric sphincter, sphincter of Oddi, and the ileocolic sphincter. These pharmaceutical preparations are also useful in treating conditions resulting from spasms and/or hypertonicity of sphincters of the anorectal region including anal fissure, post-operative rectal pain, hypertrophic pyloric stenosis, and pancreatitis, as well as conditions resulting from general spasm of the muscles of the GI tract including Zenkers diverticulum, achalasia, esophageal spasm (nutcracker esophagus), irritable bowel disease, and Hirshprungs disease (bowel obstruction).

Similarly, the invention provides methods of using the compositions in a pharmaceutically acceptable dosage form as an effective treatment for a medical condition such as hemorrhoidal pain, both before and after hemorrhoidectomy, and for treating hypertonicity and/or spasms of the sphincters including the internal anal sphincter, lower esophageal sphincter, pyloric sphincter, sphincter of Oddi, and the ileocolic sphincter. These pharmaceutical preparations are also useful in treating conditions resulting from spasms and/or hypertonicity of sphincters of the anorectal region including anal fissure, post-operative rectal pain, hypertrophic pyloric stenosis, and pancreatitis, as well as conditions resulting from general spasm of the muscles of the GI tract including Zenkers diverticulum, achalasia, esophageal spasm (nutcracker esophagus), irritable bowel disease, and Hirshprungs disease (bowel obstruction). In another aspect, the present invention provides methods for treating anal disorders, which comprise topically administering an effective amount of such composition to a subject in need of such treatment.

The methods described herein are also applicable to the treatment of recurrent anal diseases, and are also useful for relaxing the anal sphincter and reducing pain during anorectal exams or surgery (in patients with and without disorders), particularly when instruments are inserted into the anus. For example, an ointment composition of the invention can be applied topically at each application to the external anus and to the distal anal canal with the finger or an applicator. As an illustrative alternative, the medication can be delivered intra-rectally as a suppository. The medication can be applied in this fashion, for example, one or more times daily in the case of the ointment or once or more times daily in the case of the suppository.

Of course, the actual preferred course of therapy can vary according to, inter alia, the mode of administration of nitroglycerin, the particular formulation of nitroglycerin being used, the particular disease being treated, and the particular host being treated. The optimal course of therapy for a given set of conditions can be ascertained by those skilled in the art using a conventional course of therapy determination tests and in view of the information set out herein. The effectiveness of treatment can be determined by controlled clinical trials, by methods known to those of skill in the art. In particular, by methods described in the Example section.

The following examples are offered for purposes of illustration. They are intended neither to define nor to limit this invention in any manner.

### EXAMPLES

### Example 1

This example illustrates the clinical efficacy of 0.4% by weight compositions of nitroglycerin in reducing pain associated with anal conditions.

A. Methods

Three hundred and four patients at 17 centers with chronic anal fissures (single fissure present for >30 days) were randomized in a double-blind manner to one of the eight treatment regimens (0.0, 0.1, 0.2, 0.4% NTG applied b.i.d or t.i.d), for up to eight weeks. Exclusion criteria included presence of other anal pathology (fistula, abscess, recent anal surgery, etc.), Class IV cardiovascular disease, chronic NSAIDS therapy, and pregnancy. A dose-measuring device standardized the delivery of 375 mg ointment (Anogesic® NTG ointment, Cellegy Pharmaceuticals). This was applied to the distal anal canal and anus with a finger cot on the patient's finger. Drug delivery was audited by weighing of the study medication prior to distribution and on return at two weeks. Every two weeks a new tube was distributed and healing of fissures (complete re-epithelialization) was accessed by physical examination utilizing an observer unaware of treatment allocation. Patients documented their anal pain (worst pain with bowel movement, worst pain of the day and average pain) each day using a 100 mm visual analog scale (VAS). Statistical analysis for healing was accomplished with survival analysis (Cox regression and Life tables) and for pain with a generalized inbred-effects regression model.

B. Results

Patient demographics and fissure characteristics are listed in Table 1. The treatment groups were comparable for age and gender. Thirty-four patients (11%) dropped out of the study prior to healing or eight weeks. Evaluation of the remaining patients reached no significant differences in fissure healing among any of the treatment groups (Table 2; all groups, including placebo had a healing rate of approximately 50%). Treatment with 0.4% NTG ointment was associated with a highly significant (p<0.0002) decrease in pain intensity accessed by patients with visual analog scale (Table 3). The decreases were observed within the first week of treatment. Treatment was well tolerated, with only 3.6% of patients discontinuing treatment due to headache.

TABLE 1
Patient Demographics (n = 304)

| | Mean (median) | Range |
|---|---|---|
| Age | 42 years | 19-81 years |
| Gender | Female = 45.4% | |
| Duration of Symptoms | 854 (201) days | 25-11, 130 days |
| Size of Fissure | 1.4 (1.0) cm | 0.1-10 cm |
| Location | Anterior = 17% | |
| | Posterior = 80% | |
| | Anterior & Posterior = 3% | |

TABLE 2
Observed Healing Rates

| | 0.0 | 0.1 | 0.2 | 0.4 |
|---|---|---|---|---|
| All Patients (n = 304) | | | | |
| BID | 50% | 31% | 26% | 39% |
| TID | 47% | 49% | 41% | 48% |
| Completers (n = 241) | | | | |
| BID | 59% | 55% | 35% | 47% |
| TID | 53% | 55% | 47% | 67% |
| Evaluable (n = 270) | | | | |
| BID | 53% | 38% | 32% | 42% |
| TID | 52% | 53% | 42% | 59% |
| Severe - Base AVG ≧25 (n = 140) | | | | |
| BID | 38% | 43% | 14% | 29% |
| TID | 60% | 50% | 33% | 50% |
| Compliance - ≧70% (n = 167) | | | | |
| BID | 55% | 41% | 37% | 44% |
| TID | 60% | 58% | 59% | 62% |
| Duration - >100 (n = 210) | | | | |
| BID | 52% | 24% | 28% | 41% |
| TID | 54% | 50% | 40% | 46% |

TABLE 3
Percent Pain Decrease from Baseline

| | All Subjects | | | | Baseline AVG >25 mm | | | |
|---|---|---|---|---|---|---|---|---|
| Day | 0.0% | 0.1% | 0.2% | 0.4% | 0.0% | 0.1% | 0.2% | 0.4% |
| 7 | 25 | 42 | 42** | 40** | 32 | 37 | 52** | 44* |
| 14 | 42 | 46 | 46 | 49** | 46 | 43 | 55* | 58* |
| 21 | 39 | 51* | 51* | 58*** | 47 | 55 | 60* | 65** |
| 28 | 52 | 58 | 58* | 60*** | 55 | 53 | 65* | 68** |
| 35 | 50 | 57 | 57* | 66*** | 57 | 59 | 66 | 75*** |
| 42 | 54 | 63 | 63 | 65*** | 56 | 60 | 70 | 71** |
| 49 | 54 | 67 | 67** | 69*** | 57 | 67 | 74* | 78** |
| 56 | 51 | 65 | 65** | 72*** | 57 | 66 | 76** | 80*** |

Defecation Pain

| | | | | | | | | |
|---|---|---|---|---|---|---|---|---|
| 7 | 42 | 41 | 43 | 51 | 38 | 37 | 49 | 53 |
| 14 | 55 | 43 | 44 | 59 | 46 | 40 | 44 | 60 |
| 21 | 53 | 56 | 47 | 64** | 50 | 54 | 46 | 76 |
| 28 | 57 | 60 | 35 | 68** | 46 | 58 | 58 | 68* |
| 35 | 58 | 62 | 58 | 72*** | 52 | 58 | 62 | 77*** |
| 42 | 61 | 65 | 61 | 72** | 53 | 62 | 66 | 72** |
| 49 | 61 | 65 | 66 | 77*** | 52 | 64* | 74** | 81*** |
| 56 | 61 | 67 | 67 | 80*** | 55 | 65 | 78* | 83** |

Worst Pain

| | | | | | | | | |
|---|---|---|---|---|---|---|---|---|
| 7 | 39 | 39 | 46 | 48* | 39 | 38 | 49 | 48 |
| 14 | 55 | 46 | 52 | 59* | 49 | 47 | 53 | 59 |
| 21 | 56 | 60 | 56 | 65*** | 52 | 62 | 59 | 68* |
| 28 | 61 | 61 | 61 | 71*** | 55 | 58 | 65 | 73** |
| 35 | 62 | 66 | 61 | 74*** | 52 | 63 | 66 | 78** |
| 42 | 64 | 66 | 67 | 74*** | 58 | 65 | 72* | 74** |
| 49 | 61 | 71 | 70* | 77*** | 57 | 71 | 77** | 79** |
| 56 | 60 | 71 | 69 | 79*** | 57 | 70 | 79** | 82* |

*P <0.05 AVG = average
**p <0.01 Significance levels based on mixed-model
***P <0.001 Applied to raw data for intent to treat sample
++from visual Analog Scale where 0 = no pain and 100 mm = worst pain ever

C. Discussion

The proposed mechanism of action for nitroglycerin is reduced anal sphincter pressure or a primary or secondary increase in anal blood flow. Several studies have evaluated NTG for the treatment of anal fissures (Table 4). However, neither the exact dosage of drug provided to the patient nor the vehicle for delivery has been consistent or controlled in these studies.

The healing rate of chronic fissures in the placebo group from controlled studies has ranged from 8-32%. Potential reasons for the high rate of placebo healing in this study may include inadvertent inclusion of acute fissures, some therapeutic effect of the ointment vehicle, intensive medical therapy, length of follow-up, or unequal patient drop out.

The analysis of the present data indicates a highly significant decrease in pain in the 0.4% NTG group. The decrease compared to vehicle treatment was evident as early as following one week of treatment. Unexpectedly, only 3.6% of patients discontinued treatment due to headache.

It is understood that the embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes in their entirety.

### Example 2

This example illustrates a formulation of a physically stable nitroglycerin composition.

A. Manufacture of Stable Formulations

Nitroglycerin is prepared as an approximately 10% solution in propylene glycol. Sufficient nitroglycerin in propylene glycol (2 to 10%) with selected non-ionic surfactant is warmed to 40 to 60° C. and is added to a 50° C. melted mixture of lanolin/petrolatum and paraffin wax where the lanolin ranges from 0 to 25% of the final mixture. The phases are homogenized until a uniform dispersion occurs and the resulting product cooled to 20° to 30° C. while homogenizing. The product is then filled into suitable containers.

| Composition and Ranges | | |
|---|---|---|
| Ingredient | % | Range (5) |
| 10% Nitroglycerin in Propylene Glycol | 2 | 0.5 to 10 |
| Petrolatum | 75 | 60 to 90 |
| Lanolin, anhydrous | 14 | 0 to 30 |
| Sorbitan Sesquioleate* | 2 | 0.5 to 10 |
| Paraffin Wax | 5 | 1 to 10 |

*Alternate surfactants suitable, alone or as mixtures, would be sorbitan monostearate, propylene glycol monolaurate, sorbitan mono-oleate, glycerol monostearate, propylene glycol monostearate, sorbitan tristearate, and sorbitan trioleate.

B. Compositions of Different Formulations

TABLE 4

| | CLINICAL FORMULATIONS (% w/w) | | | |
|---|---|---|---|---|
| INGREDIENT | | | | |
| 10% | PLACEBO | 0.1% | 0.2% | 0.4% |
| Nitroglycerin in | 0.00 | 1.05 | 2.10 | 4.20 |
| Propylene | | | | |
| Glycol* USP | | | | |
| Propylene | 4.00 | 2.95 | 1.90 | 0.00 |
| Glycol, USP | | | | |
| White | 75.00 | 75.00 | 15.00 | 74.80 |
| Petrolatum, USP | | | | |
| Lanolin, USP/NF | 14.00 | 14.00 | 14.00 | 14.00 |
| Sorbitan | 2.00 | 2.00 | 2.00 | 2.00 |
| Sesquloleate, NF | | | | |
| Praffin Wax, NF | 5.00 | 5.00 | 5.00 | 5.00 |
| TOTAL | 100.00 | 100.00 | 100.00 | 100.00 |

*5% Overage is required due to nitroglycerin loss during manufacturing

C. Stability Data

Formulation A: Percutol (2% NTG) diluted with petrolatum to 0.2% NTG (as used in the examples in Gorfine et al.)

Formulation B: 0.4% NTG formulation

Formulation C: 0.2% NTG formulation

Formulation D: 0.2% formulation (specifically manufactured at 0.2%, not diluted from 2% ointment)

TABLE 5

| | Visual assessment of | Visual assessment of |
|---|---|---|
| | physical stability after | physical stability after |
| Formulation | 4 weeks at 40° C. | 3 months at 40° C. |
| A | Phase Separated | Not done |
| B | Not Phase Separated | Not Phase Separated |
| C | Not Phase Separated | Not Phase Separated |
| D | Phase Separated | Not done |

Advantageously, Formulations B and C remained as a single phase after 3-months of storage. Formulations A and D were biphasic after 4 weeks and are thus unsuitable for topical application do to their instability.

Current marketed formulations use nitroglycerin in ethanol or nitroglycerin adsorbed onto lactose. Formulation using nitroglycerin in ethanol cause potency increase during the processing due to evaporation of ethanol. Formulations using nitroglycerin adsorbed onto lactose produced products that are physically unstable at elevated temperature.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for relieving pain in a mammal associated with an anal condition, said method comprising:
contacting an anal area with a composition comprising nitroglycerin in an amount of 0.4% by weight, thereby relieving pain associated with said anal condition.

2. The method of claim 1, wherein said anal area is the anal canal.

3. The method of claim 1, wherein said anal condition is a member selected from the group consisting of a chronic anal fissure, an anal ulcer, hemorrhoids, a levator spasm and post-hemorrhoidectomy pain.

4. The method of claim 3, wherein said anal condition is a chronic anal fissure.

5. The method of claim 3, wherein said anal disease is post-hemorrhoidectomy pain.

6. The method of claim 1, wherein said composition further comprises an effective amount of a corticosteroid.

7. The method of claim 6, wherein said corticosteroid is present in an amount of from about 0.001% to about 10% by weight, based upon the total weight of said composition.

8. The method of claim 6, wherein said corticosteroid is a member selected from the group consisting of hydrocortisone, cortisol, and dexamethasone phosphate.

9. The method of claim 1, wherein said composition further comprises an effective amount of a topical anesthetic.

10. The method of claim 9, wherein said topical anesthetic is present in an amount of from about 0.1% to about 10% by weight, based upon the total weight of said composition.

11. The method of claim 9, wherein said anesthetic is a member selected from the group consisting of dibucaine, lidocaine, pramoxine, benzocaine, and tetracaine.

12. The method of claim 1, wherein said composition is adapted for topical administration to the external anus and the anal canal.

13. The method of claim 12, wherein said composition is adapted for topical administration to the anal canal.

14. The method of claim 1, wherein said composition comprises one or more pharmaceutically acceptable carriers or excipients in admixture with said nitroglycerin.

15. The method of claim 14, wherein said excipients comprise one or more excipients selected from the group consisting of white petrolatum, mineral oil, lanolin, distilled water, acetone sodium bisulfite, zinc oxide, and cocoa butter.

16. The method of claim 1, wherein said composition is applied to the anal canal at least one time daily.

17. The method of claim 1, wherein said composition is applied to the anal canal from 2 to 8 times daily.

18. The method of claim 1, wherein said composition is in a suitable topical form selected from the group consisting of a powder, an aerosol, a liquid, a thickened liquid, a tablet, a capsule, a semi-solid, an emulsion, an ointment, a gel, a cream, and a suppository.

19. The method of claim 18, wherein said composition is an ointment.

20. The method of claim 18, wherein said composition is a suppository.

21. The method of claim 1, wherein said composition further comprises propylene glycol and a non-ionic surfactant.

22. The method of claim 21, wherein said non-ionic surfactant is a member selected from the group consisting of sorbitan sesquioleate, sorbitan monostearate, propylene glycol monolaurate, sorbitan mono-oleate, glycerol monostearate, propylene glycol monostearate, sorbitan tristearate, and sorbitan trioleate.

23. The method of claim 22, wherein said non-ionic surfactant is sorbitan sesquioleate.

24. A method for relieving pain in a mammal associated with an anal condition, said method comprising:
contacting an anal area with a composition comprising nitroglycerin in an amount of about 150 milligrams, thereby relieving pain associated with said anal condition.

25. A topical formulation, said formulation comprising:
0.4 percent by weight nitroglycerin, propylene glycol, and a non-ionic surfactant.

26. The topical formulation of claim 25, wherein said non-ionic surfactant is a member selected from the group consisting of sorbitan sesquioleate, sorbitan monostearate, propylene glycol monolaurate, sorbitan mono-oleate, glycerol monostearate, propylene glycol monostearate, sorbitan tristearate, and sorbitan trioleate.

27. The stable formulation of claim 26, wherein said non-ionic surfactant is sorbitan sesquioleate.

28. A kit comprising: a 0.4 percent by weight nitroglycerin composition, a container, directions for use, a measuring device, and optionally an applicator.
